(19) United States
(12) Patent Application Publication
(10) Pub. No.: US 2023/0174976 A1
(43) Pub. Date: Jun. 8, 2023

(54) TARGETED RNA EDITING USING ENGINEERED ADENOSINE DEAMINASE ACTING ON TRNA (ADAT) SYSTEMS

(71) Applicant: Shape Therapeutics Inc., Seattle, WA (US)

(72) Inventors: Yiannis SAVVA, Seattle, WA (US); Richard Thomas Sullivan, Seattle, WA (US)

(21) Appl. No.: 17/919,094

(22) PCT Filed: Apr. 15, 2021
(86) PCT No.: PCT/US21/27552

§ 371 Date: Oct. 14, 2022

Related U.S. Application Data
(60) Provisional application No. 63/010,629, filed on Apr. 15, 2020.

(51) Int. Cl. C12N 15/11; A61K 31/7088; A61P 25/28

(52) U.S. Cl. CPC C12N 15/11; A61K 31/7088; A61P 25/28; C12N 2310/11; C12N 2310/3519; C12N 2320/34; C12N 2750/14143

(57) ABSTRACT

Provided herein are compositions and methods relating to providing or engineering a structural target to attract ADAT editing to a desired site. Further provided herein are compositions and methods relating to recombinant adenosine deaminase acting on tRNA (ADAT) guide tRNAs (adat-gtRNA). In certain embodiments, such compositions and methods will be useful for modifying a coding sequence of a desired protein. Also provided are methods of treating a disease or disorder associated with loss of wild-type protein expression.

### 1. CROSS-REFERENCE TO RELATED APPLICATIONS

This application claims the benefit of and priority to U.S. Provisional Patent Application No. 63/010,629 filed on Apr. 15, 2020, which is incorporated by reference in its entirety.

### 2. SEQUENCE LISTING

The instant application contains a Sequence Listing with XX sequences, which has been submitted via EFS-Web and is hereby incorporated herein by reference in its entirety. Said ASCII copy, created on XXXX, is named 45740WO_sequencelisting.txt, and is XXX bytes in size.

### 3. BACKGROUND

RNA editing is a post-transcriptional process that recodes hereditary information by changing the nucleotide sequence of RNA molecules (Rosenthal, J Exp Biol. 2015 June; 218(12): 1812-1821). Post-transcriptional RNA editing is classified into three distinct types. The first type results in insertions or deletions (indels) by addition of new, or removal of existing, nucleotides in transcripts. The second type involves nucleotide substitutions, which are generated in situ by either deamination or transamination, most commonly pyrimidine exchange—i.e., cytidine (C) to uridine (U) (C-to-U), uridine (U) to cytidine (C) (U-to-C), and adenosine-to-inosine (A-to-I) deamination. Reverse transcriptases read inosines in RNA as guanosines, and the translation machinery is thought to interpret inosines in mRNA as guanosines, potentially altering amino acid incorporation (Moreira, S. et al. Nucleic Acids Res. 2016, June 2; 44(10): 4907-4919). In RNA structural motifs/domains, A-to-I editing can influence RNA structural stability, which can modulate greater RNA stability, localization, turnover, processing post-transcriptional modifications and expression. For tRNAs, A-to-I deamination is mediated exclusively by ADAT proteins, ADAT1, ADAT2 and/or ADAT3. Deamination of tRNAs had been traditionally classified as a nucleotide modification, but is now widely described as RNA editing. The third type of RNA editing causes a nucleotide substitution as well, but acts exclusively on the 5′ and 3′ ends of the acceptor stem of mitochondrial tRNAs. In this case, mis-paired nucleotides are removed from one side of the helix and replaced by ones matching the complementary portion of the helix.

While the biological functions of endogenous RNA editing remain to be further elucidated, redirecting this endogenous machinery to engineer desired site-specific edits in target RNA species would be an attractive therapeutic modality. (Katrekar et al., Nature Methods 16(3):239-242 (2019)). Thus, further methods and systems for controlling site-specific RNA editing are needed.

### 4. SUMMARY

In an aspect, the present disclosure provides a recombinant adenosine deaminase acting on tRNA (ADAT) guide tRNA (adat-gtRNA), comprising: an ADAT recruiting domain comprising a plurality of loops of a mammalian tRNA, and a 5′ and/or a 3′ RNA targeting domain, wherein at least one RNA targeting domain has a sequence that is only partially complementary to the sequence of a segment of a target RNA, wherein binding of the adat-gtRNA to the target RNA is capable of recruiting ADAT to deaminate one or more mismatched adenosine residues in the adat-gtRNA:target RNA duplex.

In certain embodiments, the ADAT recruiting domain comprises the D loop, anticodon loop, and T loop of a mammalian tRNA. In certain embodiments, the adat-gtRNA recruiting domain lacks the 3′ amino acid attachment site and RNase cleavage sites of a mammalian tRNA acceptor stem. In certain embodiments, the ADAT recruiting domain has a structure that is capable of forming a complex with ADAT1, ADAT2, ADAT3, or any combination thereof.

In certain embodiments, the adat-gtRNA comprises a 5′ RNA targeting domain. In certain embodiments, the adat-gtRNA comprises a 3′ RNA targeting domain. In certain embodiments, the adat-gtRNA comprises both a 5′ RNA targeting domain and a 3′ RNA targeting domain. In certain embodiments, the RNA targeting domain comprises only 1 mismatched ribonucleotide that is noncomplementary to the sequence of the targeted segment of the target RNA. In certain embodiments, the RNA targeting domain comprises a plurality of mismatched ribonucleotides that are noncomplementary to the sequence of the targeted segment of the target RNA. In certain embodiments, binding of the adat-gtRNA to the target RNA induces the mismatched region of target RNA to adopt a conformation that mimics secondary structural elements of an ADAT recruiting domain of a wt-tRNA. In some embodiments, since ADAT can bind to an anticodon loop, the target A to be edited may be in an anticodon loop formed upon hybridization of an adat-gtRNA of the present disclosure to the target RNA. For example, the adat-gtRNA may comprise a first section that hybridizes to a first section of a target RNA and a second section that hybridizes to a second part of a target RNA, where the first and second sections of the target RNA are not contiguous. Thus, a loop of unpaired bases form in the target RNA upon hybridization of the adat-gtRNA to the target RNA. This loop may have a structure similar to the anticodon loop bound by ADAT and may have the target A to be edited. In other embodiments, the target A is base paired to the adat-gtRNA in the resulting duplex formed upon hybridization of the adat-gtRNA to the target RNA. In some cases, the target A is not base paired to the adat-gtRNA upon hybridization of the adat-gtRNA to the target RNA. In certain embodiments, at least one RNA targeting domain is between about 20 to about 100 nucleotides in length. In certain embodiments, the adat-gtRNA further comprises a modification at the 5′ and/or 3′ end. In certain embodiments, the target RNA is pre-mRNA or mRNA. In certain embodiments, the target RNA is a non-coding RNA. In certain embodiments, the target RNA comprises a mutation that results in alteration of wild-type protein expression. In certain embodiments, the target RNA comprises a point mutation, optionally wherein the point mutation results in a missense mutation, splice site alteration, or a premature stop codon.

In certain embodiments, the adat-gtRNA has a sequence selected from the group of sequences provided in Table 1.

In an aspect, the present disclosure provides a composition comprising at least one recombinant guide adat-gtRNA of any one of the preceding claims, and optionally a pharmaceutically acceptable excipient.

In an aspect, the present disclosure provides a vector, wherein the vector comprises a coding region, wherein the coding region encodes at least one recombinant guide adat-gtRNA of any one of the preceding claims. In certain embodiments, the vector further comprises expression control elements operably linked to the adat-gtRNA coding region. In certain embodiments, the vector is an AAV or lentiviral vector. In certain embodiments, the vector is an AAV vector.

In certain embodiments, the adat-gtRNA coding region is operably linked to expression control elements, and the expression control elements and coding region are together flanked by 5′ and 3′ AAV inverted terminal repeats (ITR). In certain embodiments, the vector packaged into a virion. In certain embodiments, the vector is formulated in a nanoparticle.

In an aspect, the present disclosure provides a method for modifying the sequence of a target RNA, comprising: contacting the target RNA with any of the recombinant guide adat-gtRNA described herein, or the composition, or a guide adat-gtRNA expressed from any of the vectors described herein.

In certain embodiments, the method further comprises delivering at least one polynucleotide, the at least one polynucleotide encoding ADAT1, ADAT2, ADAT3, or any combination thereof. In certain embodiments, the contacting is in vitro or in vivo. In certain embodiments, the method further comprises administering any of the vectors described herein to a mammalian subject in whom editing of the target RNA is desired.

In an aspect, the present disclosure provides a method of treating a disease or disorder resulting from the alteration of wild-type expression of a protein, comprising:

delivering an effective amount of at least one recombinant guide adat-tRNA described herein, to cells of a patient having a disease or disorder resulting from the loss of wild-type expression of a protein, wherein the recombinant guide adat-gtRNA is capable of recruiting ADAT to edit an RNA target, thereby increasing expression of the protein whose expression is decreased or lost. Further, delivering an effective amount of at least one recombinant guide adat-tRNA that acts on a target RNA to ameliorate a disease, regardless of changes to protein homeostasis.

In certain embodiments, the recombinant guide adat-gtRNA is delivered by administering any of the vectors described herein. In certain embodiments, the target RNA encodes the protein whose expression is decreased or lost. In certain embodiments, the subject is a human.

In an aspect, the present disclosure provides the use of an effective amount of one or more of the recombinant guide adat-gtRNAs described herein, or the composition described herein, or any of the vectors described herein, for treating a disease or disorder associated with alteration of wild-type protein expression. In certain embodiments, such compositions and methods will be useful for modifying a coding sequence of a desired protein. In certain embodiments, such compositions and methods will be useful for modifying non-coding regions such as splice sites for various manipulations such as exon skipping. Furthermore, compositions and methods of the present disclosure may be used to modify 3′UTR and/or 5′ UTR sites for regulating gene expression.

In an aspect, the present disclosure provides a kit, comprising: one or more the recombinant guide adat-gtRNAs described herein, or any of the vectors described herein, and optionally instructions for use.

In certain embodiments, the recombinant guide adat-gtRNA is hybridized to a complementary target ribonucleotide under conditions of high stringency. In certain embodiments, the recombinant guide adat-gtRNA, hybridizes to a complementary target ribonucleotide under conditions of high stringency equal to or greater than 37° C.

### 5. BRIEF DESCRIPTION OF THE DRAWINGS

FIG. 1 is a schematic showing hydrolytic deamination of adenine to inosine, the reaction catalyzed by ADAT editing.

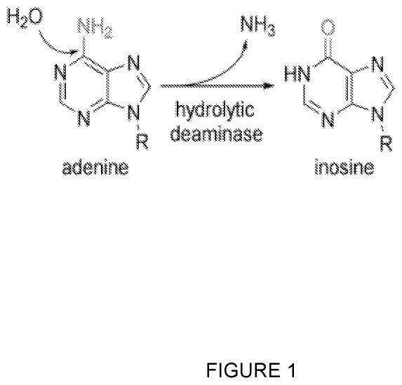

FIGS. 2A-2C represent a schematic showing tRNA recruitment of ADAT to induce an RNA edit, with FIG. 2A illustrating structural features of a native mammalian tRNA, FIG. 2B describing structural alterations to create an adat-gtRNA, and FIG. 2C illustrating binding of various adat-gtRNAs to target RNAs. As shown at the bottom of FIG. 2C, in some cases the target A to be edited may sit in an internal loop that mimics the structure of the anticodon loop of a tRNA. The internal loop is formed on the target RNA side upon hybridization of an adat-gtRNA to the target RNA.

### 6. DETAILED DESCRIPTION

We have developed a class of recombinant RNA guide molecules modeled on mammalian tRNA structures that are capable of recruiting ADAT enzymes to edit desired RNA targets. The recombinant guide molecules are capable of recruiting native ADAT1, ADAT2, and/or ADAT3 enzymes to modify/change a specific adenosine to inosine in a target RNA.

This approach provides numerous benefits over other gene editing systems. The ADAT system is found in all tissues, unlike the ADAR editing system, which is tissue-specific. This cellular ubiquity offers the possibility of relying on endogenous ADAT expression to effect the desired editing, although concomitant expression of recombinant ADAT enzymes is also contemplated. Further, ADAT expression is not induced by interferon. In contrast, ADAR expression can be induced by interferon, which may result in increased ADAR off-target editing. ADAT enzymes are highly specific to cognate tRNA secondary structure, with no evidence of promiscuous ADAT editing activity. Additionally, there is no editing of the genome when utilizing the ADAT system.

### 6.1. Definitions

Unless defined otherwise, all technical and scientific terms used herein have the meaning commonly understood by one of ordinary skill in the art to which the invention pertains.

As used herein, unless otherwise dictated by context “nucleotide” or “nt” refers to ribonucleotide.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

A “therapeutically effective amount” of a composition is an amount sufficient to achieve a desired therapeutic effect, and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein “preventing” a disease refers to inhibiting the full development of a disease.

### 6.2. adat-gtRNA

In a first aspect, recombinant adenosine deaminase acting on tRNA (ADAT) guide tRNAs are provided (adat-tRNA). The adat-gtRNA comprises an ADAT recruiting domain and at least one specific RNA targeting domain (complementary to only one target). The ADAT recruiting domain mimics the ADAT recruiting domain of a mammalian tRNA. The RNA targeting domain is at the 5′ and/or 3′ end of the recruiting domain. At least one RNA targeting domain has a sequence that is only partially complementary to the sequence of segment of the target RNA. Binding of the adat-gtRNA to the target RNA recruits ADAT enzymes to deaminate one or more mismatched adenosine residues in the adat-gtRNA RNA targeting domain:target RNA duplex.

### 6.2.1. ADAT Recruiting Domain

The ADAT recruiting domain of the recombinant adat-gtRNA mimics the ADAT-recruiting structure of a mammalian tRNA. The recruiting domain thus serves, in typical embodiments, to recruit ADAT 1, ADAT2, and/or ADAT3, or any combination thereof, to the target sequence, and facilitates subsequent editing.

In typical embodiments, the ADAT recruiting domain comprises the ADAT-recruiting structure of a mammalian tRNA. In some embodiments, the ADAT recruiting domain comprises the ADAT-recruiting structure of a tRNA from non-human organisms (e.g., yeast).

In various embodiments, the ADAT recruiting domain of the recombinant adat-gtRNA comprises the ADAT-recruiting structure of a mammalian nuclear tRNA selected from the group consisting of an arginine tRNA, an alanine tRNA, an isoleucine tRNA, a leucine tRNA, a valine tRNA, a serine tRNA, and a threonine tRNA. In some embodiments, the tRNA lacks an adenosine in the anticodon loop. In some embodiments, the tRNA ADAT recruiting domain contains one or more A-to-G mutations that serve to bind and trap ADAT on the anticodon loop. In particular embodiments, the tRNA is a human tRNA. In particular embodiments, the tRNA is a human arginine tRNA. In particular embodiments, the tRNA is a human alanine tRNA. In particular embodiments, the tRNA is a human isoleucine tRNA. In particular embodiments, the tRNA is a human leucine tRNA. In particular embodiments, the tRNA is a human valine tRNA. In particular embodiments, the tRNA is a human serine tRNA. In particular embodiments, the tRNA is a human threonine tRNA.

In various embodiments, the ADAT recruiting domain comprises a plurality of loops of a mammalian tRNA. In certain embodiments, the ADAT recruiting domain comprises 2 loops of a mammalian tRNA. In certain embodiments, the ADAT recruiting domain comprises 3 loops of a mammalian tRNA. In currently preferred embodiments, the ADAT recruiting domain comprises the D loop, anticodon loop, and T loop of a mammalian tRNA, and the ribonucleotides therebetween. In currently preferred embodiments, the ADAT recruiting domain retains the clover leaf secondary structure of a mammalian tRNA.

In certain embodiments, the ADAT recruiting domain contains the entirety of a mammalian tRNA, but with ribonucleotides at the 5′ and/or 3′ end removed, creating new, non-natural, 5′ and 3′ ends to which the 5′ RNA targeting domain, 3′ RNA targeting domain, or both 5′ and 3′ RNA targeting domains are respectively fused. The trimming of the 5′ and/or 3′ end of the native tRNA prevents or decreases RNase cleavage and provides improved stability for the recombinant construct. In addition, trimming of the 5′ and/or 3′ end of the native tRNA prevents or decreases charging of the adat-gtRNA with an amino acid.

The number of ribonucleotides to be trimmed from the 5′ and/or 3′ ends of the tRNA can range from about 1-12, 2-11, 3-10, 4-9, or 5-8 ribonucleotides. A currently preferred range of nucleotides to be trimmed is from 3-5. In various embodiments, 1 nt, 2 nt, 3 nt, 4 nt, 5 nt, 6 nt, 7 nt, 8 nt, or 9 nt are deleted from the 5′ end of the tRNA. In various embodiments, 1 nt, 2 nt, 3 nt, 4 nt, 5 nt, 6 nt, 7 nt, 8 nt, or 9 nt are deleted from the 3′ end of the tRNA. In various embodiments, 1 nt, 2 nt, 3 nt, 4 nt, 5 nt, 6 nt, 7 nt, 8 nt, or 9 nt are independently deleted from the 5′ end and 3′ end of the tRNA. The number of nucleotides trimmed from the 5′ and/or 3′ end of the tRNA may permissibly be any number that will maintain the desired ADAT recruiting ability of the recombinant guide adat-gtRNA.

In various embodiments, the recruiting domain is between 40-90 ribonucleotides in length. In some embodiments, the recruiting domain is between 50-80 ribonucleotides in length, or 60-70 ribonucleotides in length. In certain embodiments, the recruiting domain is 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, or 90 nt in length.

In various embodiments, the ADAT recruiting domain comprises the ADAT-recruiting structure of a mammalian tRNA with one or more substitutions, insertions and/or deletions of nucleotides, so long as the ADAT recruiting activity is not lost. In some embodiments, the one or more substitutions, insertions and deletions of nucleotides improves a desired property of the recombinant adat-gtRNA. In some embodiments, the one or more substitutions, insertions and/or deletions are chosen to correspond to residues of a different mammalian tRNA.

In some embodiments, the engineered guide adat-gtRNA will recruit any one or a combination of ADAT1, ADAT2, and ADAT3. In some embodiments, the engineered guide adat-gtRNA will have preferential binding to ADAT1. In some embodiments, the engineered guide adat-gtRNA will have preferential binding to ADAT2. In some embodiments, the engineered guide adat-gtRNA will have preferential binding to ADAT3.

The engineered guide adat-gtRNA can comprise a plurality of ADAT recruiting domains. In typical embodiments, the adat-gtRNA has one ADAT recruiting domain. In some embodiments, the adat-gtRNA has two ADAT recruiting domains. In certain embodiments, each of a plurality of ADAT recruiting domains assumes the cloverleaf secondary structure of a mammalian tRNA.

### 6.2.2. RNA Targeting Domains

The recombinant adat-gtRNA comprises at least one RNA targeting domain that localizes the adat-gtRNA to the target RNA desired to be edited through sequence complementarity to a segment of the target RNA— i.e., the RNA targeting domain has a sequence that is “antisense” to the sequence of the target RNA. At least one of the at least one RNA targeting domains has a sequence that is only partially complementary to the sequence of the segment of the target RNA desired to be edited.

In some embodiments, the adat-gtRNA comprises one RNA targeting domain. The RNA targeting domain is only partially complementary to the sequence of the segment of the target RNA desired to be edited. In certain embodiments, the RNA targeting domain is at the 5′ end of the recruiting domain. In certain embodiments, the RNA targeting domain is at the 3′ end of the recruiting domain.

In some embodiments, the adat-gtRNA comprises two RNA targeting domains, one at the 5′ end of the recruiting domain and one at the 3′ end of the recruiting domain. In some embodiments, one of the two RNA targeting domains is only partially complementary to the sequence of the segment of the target RNA desired to be edited, and the other RNA targeting domain is fully complementary in sequence to the target RNA. In some embodiments, both of the RNA targeting domains are partially complementary to the sequence of the target RNA, each to a segment of the target RNA desired to be edited.

In various embodiments, the partially complementary RNA targeting domain has only 1 mismatched nucleotide that is noncomplementary to the sequence of the targeted segment of the target RNA. In various embodiments, the partially complementary RNA targeting domain has a plurality of mismatched nucleotides that are noncomplementary to the sequence of the targeted segment of the target RNA. In certain of these latter embodiments, binding of the adat-gtRNA to the target RNA induces the mismatched region of target RNA to adopt a conformation that mimics secondary structure of an ADAT recruiting domain of a wild type tRNA.

In embodiments with a single RNA targeting domain, the RNA targeting domain is of sufficient length to bind to the target RNA under intracellular conditions, notwithstanding the one or more complementarity mismatches. In preferred embodiments, the RNA targeting domain is of sufficient length to bind under intracellular conditions preferentially to the target RNA over RNA species that are not desired to be edited. In embodiments with two RNA targeting domains, the RNA targeting domains are together of sufficient length to bind to the target RNA under intracellular conditions, notwithstanding the one or more complementarity mismatches in one or both RNA targeting regions. In preferred embodiments, the RNA targeting domains are together of sufficient length to bind under intracellular conditions preferentially to the target RNA over RNA species that are not desired to be edited.

In typical embodiments, the RNA targeting domain is between about 20 to about 100 nucleotides in length. In some embodiments, the RNA targeting domain is 20-100 nt, 30-90 nt, 40-80 nt, or 50-70 nt in length. In some embodiments, the RNA target domain is at least 20, 25, 30, 35, 40, 45, 50, 55, 60, 65, 70, 75, 80, 85 or 90 nucleotides in length. In some embodiments, the RNA targeting domain is no more than 90, 80, 70, 60, 50, 40, or 30 nucleotides in length.

### 6.2.2.1 Target RNA

In some embodiments, the target RNA to be edited is a pre-mRNA. In some embodiments, the target RNA is a mature mRNA. In some embodiments, the target RNA is a non-coding RNA. In select embodiments, the target RNA is an miRNA, siRNA or lncRNA.

In certain embodiments, the target RNA is a splice acceptor or donor site. In certain additional embodiments, the target RNA is a 5′-UTR and/or a 3′-UTR.

In currently preferred embodiments, the target RNA is an mRNA and/or pre-mRNA. In some embodiments, the mRNA and/or pre-mRNA comprises a mutation that results in gain or loss of wild-type protein expression, and editing effected by contacting the target RNA with the adat-gtRNA alters expression of the protein encoded by the RNA. In some embodiments, full expression of the protein is restored or repressed. In some embodiments, partial expression is restored or repressed. In particular embodiments, sufficient expression is restored or repressed to improve signs or symptoms of a disease or disorder. In select embodiments, the target RNA is expressed from a mutated gene that causes one of the diseases described in section 4.7 below.

In certain embodiments, the target RNA comprises a point mutation. In particular embodiments, the point mutation results in a missense mutation, splice site alteration, or a premature stop codon.

### 6.2.3. Other Modifications

In some embodiments, the adat-tRNA further comprises a modification at the 5′ or 3′ end. In certain embodiments, the modification reduces exonuclease digestion of the adat-gtRNA.

In some embodiments the adat-tRNA comprises one or more non-natural nucleotides. In some embodiments, the adat-tRNA includes inter-nucleotide linkages that are not phosphodiester linkages.

### 6.3. Compositions Comprising Adat-gtRNA

The recombinant guide nucleic acid molecules described herein can be in any number of suitable forms, including in naked form, in complexed form, or in a delivery vehicle.

In certain embodiments, the recombinant adat-gtRNA guide is in naked form. In particular embodiments, the adat-gtRNA is in a fluid composition without any other carrier proteins or delivery vehicles. In certain embodiments, the recombinant adat-gtRNA is in complexed form, bound to other nucleic acid or amino acids that assist in maintaining stability, such as by reducing exonuclease or endonuclease digestion.

In some embodiments, the adat-gtRNA is formulated into a composition that comprises the adat-gtRNA and at least one carrier or excipient. In some embodiments intended for direct administration of the adat-gtRNA to a patient, the adat-gtRNA is formulated in a pharmaceutical composition that comprises the adat-gtRNA and at least one pharmaceutically acceptable carrier or excipient. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can further be incorporated into the compositions.

Delivery vehicles such as liposomes, nanocapsules, microparticles, microspheres, lipid particles, vesicles, and the like, may be used for the introduction of any of the recombinant nucleic acids or compositions described herein into suitable host cells. In particular, the compositions or recombinant nucleic acids may be formulated for delivery either encapsulated within or functionalized onto a lipid particle, a liposome, a vesicle, a nanosphere, a polymer, or a nanoparticle or the like.

Methods to deliver recombinant guide nucleic acid molecules and related compositions described herein include any suitable method including: via nanoparticles include using liposomes, synthetic polymeric materials, naturally occurring polymers and inorganic materials to form nanoparticles.

Examples of lipid-based materials for delivery of the DNA or RNA molecules include: polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trim ethyl ammonium chloride (DOTMA), N-[1-(2,3-dioleoloxy)-propyl]-N,N,N-trimethylammonium methyl sulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., ceDNA) can also be complexed with, e.g., poly (L-lysine) or avidin and lipids may, or may not, be included in this mixture, e.g., steryl-poly (L-lysine).

Naturally occurring polymers which can be used include chitosan, protamine, atelocollagen and peptides.

Inorganic materials that may also be used include gold nanoparticles, silica-based, and magnetic nanoparticles, which may be produced by methods known to the person skilled in the art.

### 6.4. Vectors Encoding Adat-gtRNA

In another aspect, vectors encoding an adat-gtRNA are provided.

In some embodiments, the vector does not express the adat-gtRNA and is used to propagate polynucleotides that encode the adat-gtRNA. In typical embodiments, the encoding polynucleotide is DNA. In some embodiments, the vector is a plasmid. In some embodiments, the vector is a phage. In some embodiments, the vector is a phagemid. In some embodiments, the vector is a cosmid.

In some embodiments, the vector is capable of expressing the adat-gtRNA. Expression vectors can be used to introduce the adat-gtRNA into cells in vitro or in vivo.

In typical expression vector embodiments, the vector comprises a coding region, wherein the coding region encodes at least one recombinant adat-tRNA as described herein. The coding region is operably linked to expression control elements that direct transcription. In some embodiments, the expression vector is an adenoviral vector, an adeno-associated virus (AAV) vector, a retroviral vector, or a lentiviral vector. In certain preferred embodiments, the vector is an AAV vector, and the expression control elements and adat-gtRNA coding region are together flanked by 5′ and 3′ AAV inverted terminal repeats (ITR). In some embodiments the AAV vector is selected from various AAV serotypes, including, but not limited to, AAV1, AAV2, AAV3, AAV4, AAV5, AAV6, AAV7, AAV8, AAV9, AAV10, AAV11, or chimeric combinations thereof.

In some embodiments, the vector is packaged into a recombinant virion. In particular embodiments, the vector is packaged into a recombinant AAV virion.

### 6.5. Compositions Comprising Adat-gtRNA-Encoding Vectors

In another aspect, compositions comprising the adat-gtRNA-encoding vectors are provided.

In some embodiments, the compositions are suitable for administration to a patient, and the composition is a pharmaceutical composition comprising a recombinant virion and at least one pharmaceutically acceptable carrier or excipient. In typical embodiments, the pharmaceutical composition is adapted for parenteral administration. In certain embodiments, the pharmaceutical composition is adapted for intravenous administration, intravitreal administration, posterior retinal administration, intrathecal administration, or intra-ci sterna magna (ICM) administration.

### 6.6. Methods for Editing RNA

To effect editing of RNA, the adat-gtRNA is contacted to the target RNA in the presence of ADAT enzymes. Typically, contact is within a cell. In certain embodiments, the contacting is performed in vitro. In certain embodiments, the contacting is performed in vivo.

Thus, in another aspect, methods are provided for editing target RNAs. The methods comprise contacting the target RNA with at least one recombinant guide adat-gtRNA as described herein. In some embodiments, the contacting is performed in vitro. In some embodiments, the contacting is performed in vivo.

In some embodiments, the method comprises the preceding step of introducing one or more adat-gtRNAs into a cell comprising the target RNA. In some embodiments, the method comprises the preceding step of introducing one or more recombinant expression vectors that are capable of expressing the one or more adat-gtRNAs into the cell. In some embodiments, the methods further comprise delivering an ADAT enzyme, or ADAT-encoding polynucleotide, into the cell.

The recombinant guide nucleic acid molecules and vectors disclosed herein can be introduced into desired or target cells by any techniques known in the art, such as liposomal transfection, chemical transfection, micro-injection, electroporation, gene-gun penetration, viral infection or transduction, transposon insertion, jumping gene insertion, and a combination thereof.

The recombinant guide nucleic acid molecules and related compositions disclosed herein can be delivered by any suitable system, including by using any gene delivery vectors, such as adenoviral vector, adeno-associated vector, retroviral vector, lentiviral vector, or a combination thereof. In some embodiments, a recombinant adenoviral vector, a recombinant adeno-associated vector, a recombinant retroviral vector, a recombinant lentiviral vector, or a combination thereof, may be used to introduce any of the recombinant guide molecules or nucleic acid molecules described herein.

In some embodiments, the recombinant guide nucleic acid molecules disclosed herein may be present in a composition comprising physiologically acceptable carriers, excipients, adjuvants, or diluents. Neutral buffered saline or saline mixed with serum albumin are exemplary appropriate diluents. Suitable carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutically acceptable carriers (vehicles) useful in this disclosure are conventional. In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

### 6.7. Methods of Treating Diseases Caused by Alteration of Wild-Type Protein Expression

In another aspect, methods are provided for treating diseases caused by loss or gain of wild-type expression. The method comprises delivering an effective amount of at least one recombinant guide adat-tRNA to a patient having a disease or disorder resulting from the loss or gain of wild-type expression of a protein, wherein the recombinant guide adat-tRNA is capable of recruiting ADAT to edit an RNA target, thereby increasing or decreasing expression of the protein whose expression is decreased or increased.

There are numerous examples of diseases or conditions caused by aberrant protein expression, or loss of wild-type protein expression (i.e. either increased or decreased from wild-type expression) that would be suitable for treatment using the methods described herein relating to ADAT editing.

An example includes conditions caused by missense mutations, which can render the resulting protein nonfunctional. Examples of such mutations that are responsible for human diseases including Epidermolysis bullosa, sickle-cell disease, and SOD1 mediated amyotrophic lateral sclerosis (ALS). Another example is cystic fibrosis (Human Molecular Genetics, Vol. 7, Issue 11, October 1998, Pages 1761-1769.).

The ADAT-mediated RNA editing of the present disclosure is useful in a plurality of cell and tissue types that exhibit aberrant protein expression. ADAT-mediated RNA editing may be useful for editing RNA of various tissues, including, but not limited to, tissue of the liver, eye, muscle, central nervous system, and other critical organ systems.

The RNA editing techniques and methods described herein are likely to be most beneficial during the newborn or infant stages, but may provide benefits at any stage of life. The term pediatric typically refers to anyone under 15 years of age, and less than 35 kg. A neonate typically refers to newborn up to first 28 days of life. The term infant typically refers to an individual from the neonatal period up to 12 months. The term toddler typically refers to an individual from 1-3 years of age. Teenagers are typically considered to be 13-19 years of age. Young adults are typically considered to be from 19-24 years of age.

### 6.8. Kits

Additionally, certain components or embodiments of the heterologous/recombinant engineered guide nucleic acid molecule compositions can be provided in a kit. For example, any of the heterologous/recombinant engineered guide nucleic acid compositions, as well as the related buffers or other components related to administration can be provided frozen and packaged as a kit, alone or along with separate containers of any of the other agents from the pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator. In some embodiments, the kits include all components needed for the stages of conditioning/treatment. In some embodiments, the compositions may have preservatives or be preservative-free (for example, in a single-use container).

### 6.9. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1

The following experiments will be carried out in order to further delineate key aspects of the ADAT gtRNAs.

A. Analysis of Recombinant gtRNA Structure and Sequences for RNase Recognition and subsequent cleavage.

Experiments to determine whether the antisense targeting sequences are retained in the test adat-gtRNAs are performed. Analysis of various amounts of trimming the tRNA RNase recognition sites, in conjunction with the attachment of test adat-gtRNAs are performed. Current data indicates that trimming the tRNA RNase recognition sites does not reduce ADAT editing activity, but instead actually increases the tRNA editing by two-fold.

B. Construct ADAT-gtRNAs to Include Antisense Sequences at Either the 5′ End, the 3′ End or at Both Ends of the Guide-tRNA.

Experiments to profile adat-gtRNA placement across a test set of tRNA-inspired structural elements will identify optimal tRNA structures and placement.

C. Evaluating Presence of Comparable Rab7a and ABCA4 Editing Compared to Established ADAR Mediated Guides.

ADAT expressing cell lines will be identified and used to assess ADAT mediated editing. Wildtype K562 and 293 cell lines will be analyzed for ADAT mediated editing in an ADAR KO cell line context. This will allow for determining ADAT involvement in the guide meditated editing (ADAT-ADAT homodimer).

D. Evaluation of ADAT KO Lines.

Experiments using ADAT knockout (KO) cell lines will be run to determine whether any individual ADAT proteins, either ADAT 1, 2 or 3, can be added to complement, i.e. restore ADAT editing in an ADAT knockout.

These experiments will provide models illustrating that the recombinant gtRNA based methods and compositions described herein will be useful for treating tissues that are low in endogenous ADAR activity, as well as useful in methods for treating diseases or conditions caused by decrease or loss of wild-type expression of a protein.

6.10. Sequences

An example of a wild-type Arginine tRNA (SEQ ID NO:1) 5′-GGGCCAGUGGCGCAAUGGAUAACGCGUCUGACUUCAGAUCAGAAGAUUCCAGG UUCGACUCCUGGCUGGCUC G-3′.

A hypothetical example of modified recombinant guide tRNA (adat tRNA) sequence containing antisense flanking sequences denoted as N: (SEQ ID NO:2) 5′-NNNNNNNNNNNNNNNNCAGUGGCGCAAUGGAUAACGCGUCUGACUUCAGAUC AGAAGAUUCCAGGUUCGACUCCUGGCUGNNNNNNNNNNNNNNNNN-3′.

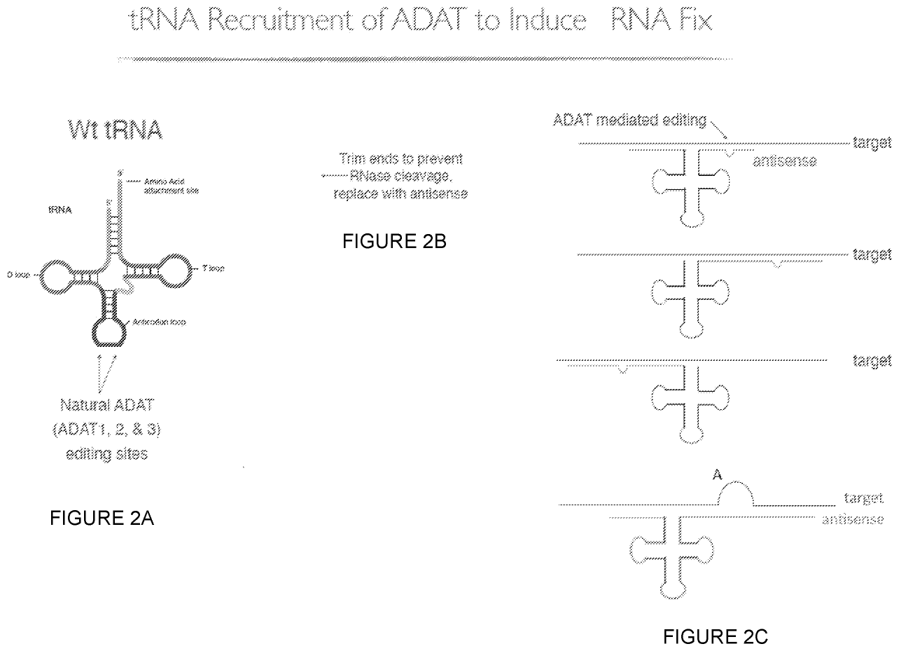

In this sequence example, four ribonucleotides have been trimmed from the 5′ flanking region and replaced with 16 replacement antisense ribonucleotides, which will serve to bind to the target RNA. Similarly, at the 3′ terminus, five ribonucleotides have been trimmed and replaced with 17 antisense ribonucleotides which will bind to the target RNA. The antisense flanking sequences when bound to the target will expose the cloverleaf structure of the modified guide tRNA, which will attract the ADAT1, 2, and/or 3 enzyme for editing (See FIGS. 2A-C).

In certain embodiments, an ADAT recruiting domain comprising a plurality of loops of a mammalian tRNA can include one or more engineered nucleotide substitutions, which maintain the desired conformation.

In certain embodiments, the binding of the adat-gtRNA to the target RNA is capable of recruiting ADAT to deaminate one or more mismatched adenosine residues in the adat-gtRNA:target RNA duplex. A schematic of an exemplary mismatched adenosine residue is shown in FIG. 2C as a “bubble” in the flanking targeting sequence of the adat-gtRNA that has bound to the target RNA, forming the adat-gtRNA:target RNA duplex.

TABLE 1

| | | SEQ |
|---|---|---|
| | | ID |
| SEQUENCE | DESCRIPTION | NO |
| GGGCCAGUGGCGCAAUGGAUAACGCGUCUGACUUCAGAUCAGAAGAUUCCAGGUUCGACU | Arginine tRNA | 1 |
| CCUGGCUGGCUCG | | |
| 5′-NNNNNNNNNNNNNNNNCAGUGGCGCAAUGGAUAACGCGUCUGACUUCAGAUCAGAAG | Modified | 2 |
| AUUCCAGGUUCGACUCCUGGCUGNNNNNNNNNNNNNNNNN-3′ | recombinant | |
| | guide tRNA | |
| MWTADEIAQLCYEHYGIRLPKKGKPEPNHEWTLLAAVVKIQSPADKACDTPDKPVQVTKE | Human ADAT1 | 3 |
| VVSMGTGTKCIGQSKMRKNGDILNDSHAEVIARRSFQRYLLHQLQLAATLKEDSIFVPGT | (Uniprot | |
| QKGVWKLRRDLIFVFFSSHTPCGDASIIPMLEFEDQPCCPVFRNWAHNSSVEASSNLEAP | Q9BU4) | |
| GNERKCEDPDSPVTKKMRLEPGTAAREVTNGAAHHQSFGKQKSGPISPGIHSCDLTVEGL | | |
| ATVTRIAPGSAKVIDVYRTGAKCVPGEAGDSGKPGAAFHQVGLLRVKPGRGDRTRSMSCS | | |
| DKMARWNVLGCQGALLMHLLEEPIYLSAVVIGKCPYSQEAMQRALIGRCQNVSALPKGFG | | |
| VQELKILQSDLLFEQSRSAVQAKRADSPGRLVPCGAAISWSAVPEQPLDVTANGFPQGTT | | |
| KKTIGSLQARSQISKVELFRSFQKLLSRIARDKWPHSLRVQKLDTYQEYKEAASSYQEAW | | |
| STLRKQVFGSWIRNPPDYHQFK | | |
| MEAKAAPKPAASGACSVSAEETEKWMEEAMHMAKEALENTEVPVGCLMVYNNEVVGKGRN | Human ADAT2 | 4 |
| EVNQTKNATRHAEMVAIDQVLDWCRQSGKSPSEVFEHTVLYVTVEPCIMCAAALRLMKIP | (Uniprot | |
| LVVYGCQNERFGGCGSVLNIASADLPNTGRPFQCIPGYRAEEAVEMLKTFYKQENPNAPK | Q7Z6V5) | |
| SKVRKKECQKS | | |
| MEPAPGLVEQPKCLEAGSPEPEPAPWQALPVLSEKQSGDVELVLAYAAPVLDKRQTSRLL | Human ADAT3 | 5 |
| KEVSALHPLPAQPHLKRVRPSRDAGSPHALEMLLCLAGPASGPRSLAELLPRPAVDPRGL | (Uniprot | |
| GQPFLVPVPARPPLTRGQFEEARAHWPTSFHEDKQVTSALAGRLFSTQERAAMQSHMERA | Q96EY9) | |
| VWAARRAAARGLRAVGAVVVDPASDRVLATGHDCSCADNPLLHAVMVCVDLVARGQGRGT | | |
| YDFRPFPACSFAPAAAPQAVRAGAVRKLDADEDGLPYLCTGYDLYVTREPCAMCAMALVH | | |
| ARILRVFYGAPSPDGALGTRFRIHARPDLNHRFQVFRGVLEEQCRWLDPDT | | |
| GGCTCGTTGGTCTAGGGGTATGATTCTCGCTTAGGATGCGAGAGGtCCCG | Proline tRNA | 6 |
| GGTTCAAATCCCGGACGAGCCC | | |
| GGTAGCGTGGCCGAGCGGTctAAGGCGCTGGATTAAGGCTCCAGTCTC | Leucine tRNA | 7 |
| TTCGGAGGCGTGGGTTCGAATCCCACCGCTGCCA | | |
| GTTTCCGTAGTGTAGTGGTtATCACGTTCGCCTAACACGCGAAAGGtC | Valine tRNA | 8 |
| CCCGGTTCGAAACCGGGCGGAAACA | | |
| GTAGTCGTGGCCGAGTGGTtAAGGCGATGGACTAGAAATCCATTGGGGT | Serine tRNA | 9 |
| TTCCCCGCGCAGGTTCGAATCCTGCCGACTACG | | |
| GGGCGUUGGCGUAGUAAGUAGCGCGCUCCCUUAGCAUGGG | Alanine tRNA | 10 |
| AGAGGUCUCCGUUCGAUUCGGAUCGUCCACCA | (yeast) | |
| GGGGGATTAGCTCAAATGGTAGAGCGCTCGCTTAGCATGCG | Alanine tRNA | 11 |
| AGAGGtAGCGGGATCGATGCCCGCATCCTCCA | | |
| GGCGCCGTGGCTTAGTTGGTtAAAGCGCCTGTCTAGTAAAC | Threonine | 12 |
| AGGAGAtCCTGGGTTCGAATCCCAGCGGTGCCT | tRNA | |
| GGCTGGTTAGTTCAGTTGGTtAGAGCGTGGTGCTAATAACG | Isoleucine | 13 |
| CCAAGGtCGTGGGTTCGATCCCCATATCGGCCA | tRNA | |

ADAT1 human sequence can be found at: UNIPROTKB:Q9BUB4, first described by Maas, S. et al. Proc. Natl. Acad. Sci. U.S.A. 96:8895-8900(1999). Similarly, the ADAT2 human sequence can be found at: UNIPROTKB:Q7Z6V5. Finally, the ADAT3 human sequence can be found at: UNIPROTKB:Q96EY9.

### 6.11. Equivalents and Incorporation by Reference

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A recombinant adenosine deaminase acting on tRNA (ADAT) guide tRNA (adat-gtRNA), comprising:
an ADAT recruiting domain comprising a plurality of loops of a mammalian tRNA, and
a 5′ and/or a 3′ RNA targeting domain,
wherein at least one RNA targeting domain has a sequence that is only partially complementary to the sequence of a segment of a target RNA,
wherein binding of the adat-gtRNA to the target RNA is capable of recruiting ADAT to deaminate one or more mismatched adenosine residues in the adat-gtRNA:target RNA duplex.

2. The adat-gtRNA of claim 1, wherein the ADAT recruiting domain comprises the D loop, anticodon loop, and T loop of a mammalian tRNA.

3. The adat-gtRNA of claim 2, wherein the adat-gtRNA recruiting domain lacks the 3′ amino acid attachment site and RNase cleavage site of a mammalian tRNA acceptor stem.

4. The recombinant guide adat-gtRNA of any one of claims 1-3, wherein the ADAT recruiting domain has a structure that is capable of forming a complex with ADAT1, ADAT2, ADAT3, or any combination thereof.

5. The recombinant guide adat-gtRNA of any one of claims 1-4, wherein the adat-gtRNA comprises a 5′ RNA targeting domain.

6. The recombinant guide adat-gtRNA of any one of claims 1-5, wherein the adat-gtRNA comprises a 3′ RNA targeting domain.

7. The recombinant guide adat-gtRNA of any one of claims 1-6, wherein the adat-gtRNA comprises both a 5′ RNA targeting domain and a 3′ RNA targeting domain.

8. The recombinant guide adat-gtRNA of any one of claims 1-7, wherein the RNA targeting domain comprises only 1 mismatched ribonucleotide that is noncomplementary to the sequence of the targeted segment of the target RNA.

9. The recombinant guide adat-gtRNA of any one of claims 1-7, wherein the RNA targeting domain comprises a plurality of mismatched ribonucleotides that are noncomplementary to the sequence of the targeted segment of the target RNA.

10. The recombinant guide adat-gtRNA of claim 9, wherein binding of the adat-gtRNA to the target RNA induces the mismatched region of target RNA to adopt a conformation that mimics secondary structure of an ADAT recruiting domain of a wt-tRNA.

11. The recombinant guide adat-gtRNA of claim 10, wherein the conformation that mimics the secondary structure of an ADAT recruiting domain of a wt-tRNA is capable of recruiting ADAT to deaminate one or more mismatched adenosine residues in the adat-gtRNA:target RNA duplex.

12. The recombinant guide adat-gtRNA of any one of claims 1-11, wherein at least one RNA targeting domain is between about 20 to about 100 nucleotides in length.

13. The recombinant guide adat-gtRNA of any one of the preceding claims, wherein the adat-gtRNA further comprises a modification at the 5′ and/or 3′ end.

14. The recombinant guide adat-gtRNA of any one of claims 1-13, wherein the target RNA is pre-mRNA or mRNA.

15. The recombinant guide adat-gtRNA of any one of claims 1-13, wherein the target RNA is non-coding RNA.

16. The recombinant guide adat-gtRNA of claim 14, wherein the target RNA comprises a mutation that results in loss of wild-type protein expression.

17. The recombinant guide adat-gtRNA of claim 14, wherein the target RNA comprises a mutation that results in gain of wild-type protein expression.

18. The recombinant guide adat-gtRNA of claim 16, wherein the target RNA comprises a point mutation, optionally wherein the point mutation results in a missense mutation, splice site alteration, or a premature stop codon.

19. The recombinant guide adat-gtRNA of any one of the preceding claims, wherein the adat-gtRNA has a sequence selected from the group of sequences provided in Table 1.

20. A composition comprising at least one recombinant guide adat-gtRNA of any one of the preceding claims, and optionally a pharmaceutically acceptable excipient.

21. A vector, wherein the vector comprises a coding region, wherein the coding region encodes at least one recombinant guide adat-gtRNA of any one of the preceding claims.

22. The vector of claim 19, further comprising expression control elements operably linked to the adat-gtRNA coding region.

23. The vector of claim 21 or claim 22, wherein the vector is an AAV or lentiviral vector.

24. The vector of claim 23, wherein the vector is an AAV vector.

25. The vector of claim 24, wherein the adat-gtRNA coding region is operably linked to expression control elements, and the expression control elements and coding region are together flanked by 5′ and 3′ AAV inverted terminal repeats (ITR).

26. The vector of any one of claims 21-25, packaged into a virion.

27. The vector of any one of claims 21-25, formulated in or on a nanoparticle.

28. A method for modifying the sequence of a target RNA, comprising:
contacting the target RNA with the recombinant guide adat-gtRNA of any one of claims 1-19, the composition of claim 20, or a guide adat-gtRNA expressed from the vector of any one of claims 21-27.

29. The method of claim 28, further comprising delivering at least one polynucleotide, the at least one polynucleotide encoding ADAT1, ADAT2, ADAT3, or any combination thereof.

30. The method of claim 28 or claim 29, wherein the contacting is in vitro or in vivo.

31. The method of claim 30, comprising:
administering the vector of any one of claims 21-27 to a mammalian subject in whom editing of the target RNA is desired.

32. A method of treating a disease or disorder resulting from the decrease or loss of wild-type expression of a protein, comprising:
delivering an effective amount of at least one recombinant guide adat-tRNA of any one of claims 1-19 to cells of a patient having a disease or disorder resulting from the loss of wild-type expression of a protein,
wherein the recombinant guide adat-gtRNA is capable of recruiting ADAT to edit an RNA target, thereby increasing expression of the protein whose expression is decreased or lost.

33. A method of treating a disease or disorder resulting from the increase or gain of wild-type expression of a protein, comprising:
delivering an effective amount of at least one recombinant guide adat-tRNA of any one of claims 1-19 to cells of a patient having a disease or disorder resulting from the increase or gain of wild-type expression of a protein,
wherein the recombinant guide adat-gtRNA is capable of recruiting ADAT to edit an RNA target, thereby decreasing expression of the protein whose expression is decreased or lost.

34. The method of claim 32 or claim 33, wherein the recombinant guide adat-gtRNA is delivered by administering a vector of any one of claims 21-27.

35. The method of any one of claims 32-34, wherein the target RNA encodes the protein whose expression is decreased is lost.

36. The method of any one of claims 32-35, wherein the subject is a human.

37. Use of an effective amount of one or more of the recombinant guide adat-gtRNAs of any one of claims 1-19, the composition of claim 20, or the vector of any one of claims 21-27, for treating a disease or disorder associated with loss of wild-type protein expression.

38. A kit, comprising:
one or more the recombinant guide adat-gtRNAs of any one of claims 1-18 or the vector of any one of claims 21-27, and
optionally instructions for use.

39. Any of the recombinant guide adat-gtRNA of claims 1-19, hybridized to a complementary target ribonucleotide under conditions of high stringency.

40. The recombinant guide adat-gtRNA of claim 39, which hybridizes to a complementary target ribonucleotide under conditions of high stringency equal to or greater than 37° C.

41. A recombinant adenosine deaminase acting on tRNA (ADAT) guide tRNA (adat-gtRNA), comprising:
an ADAT recruiting domain comprising a plurality of loops of a mammalian tRNA, and a 5′ and/or a 3′ RNA targeting domain,
wherein at least one RNA targeting domain has a sequence that is only partially complementary to the sequence of a segment of a target RNA,
wherein binding of the adat-gtRNA to the target RNA results in a RNA secondary structure that mimics the anticodon loop of a tRNA that is capable of recruiting ADAT to deaminate one or more mismatched adenosine residues in the adat-gtRNA:target RNA duplex.


SEQUENCE LISTING (13 sequences)

SEQ ID NO: 1 - RNA, 73 nt, Unknown
  Arginine tRNA sequence
gggccaguggcgcaauggauaacgcgucugacuucagaucagaagauuccagguucgacuccuggcuggcucg

SEQ ID NO: 2 - RNA, 97 nt, Artificial Sequence
  Synthetic oligonucleotide
  modified_base (1)..(16): a, c, u, g, unknown or other
  misc_feature (1)..(97): See specification as filed for detailed description of substitutions and preferred embodiments
  modified_base (81)..(97): a, c, u, g, unknown or other
nnnnnnnnnnnnnnnncaguggcgcaauggauaacgcgucugacuucagaucagaagauuccagguucgacuccuggcugnnnnnnnnnnnnnnnnn

SEQ ID NO: 3 - protein, 502 aa, Homo sapiens
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 4 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 5 - protein, 351 aa, Homo sapiens
MEPAPGLVEQPKCLEAGSPEPEPAPWQALPVLSEKQSGDVELVLAYAAPVLDKRQTSRLLKEVSALHPLPAQPHLKRVRPSRDAGSPHALEMLLCLAGPASGPRSLAELLPRPAVDPRGLGQPFLVPVPARPPLTRGQFEEARAHWPTSFHEDKQVTSALAGRLFSTQERAAMQSHMERAVWAARRAAARGLRAVGAVVVDPASDRVLATGHDCSCADNPLLHAVMVCVDLVARGQGRGTYDFRPFPACSFAPAAAPQAVRAGAVRKLDADEDGLPYLCTGYDLYVTREPCAMCAMALVHARILRVFYGAPSPDGALGTRFRIHARPDLNHRFQVFRGVLEEQCRWLDPDT

SEQ ID NO: 6 - DNA, 72 bp, Unknown
  Proline tRNA sequence
ggctcgttggtctaggggtatgattctcgcttaggatgcgagaggtcccgggttcaaatcccggacgagccc

SEQ ID NO: 7 - DNA, 82 bp, Unknown
  Leucine tRNA sequence
ggtagcgtggccgagcggtctaaggcgctggattaaggctccagtctcttcggaggcgtgggttcgaatcccaccgctgcca

SEQ ID NO: 8 - DNA, 73 bp, Unknown
  Valine tRNA sequence
gtttccgtagtgtagtggttatcacgttcgcctaacacgcgaaaggtccccggttcgaaaccgggcggaaaca

SEQ ID NO: 9 - DNA, 82 bp, Unknown
  Serine tRNA sequence
gtagtcgtggccgagtggttaaggcgatggactagaaatccattggggtttccccgcgcaggttcgaatcctgccgactacg

SEQ ID NO: 10 - RNA, 72 nt, Unknown
  Alanine tRNA (yeast) sequence
gggcguuggcguaguaaguagcgcgcucccuuagcaugggagaggucuccguucgauucggaucguccacca

SEQ ID NO: 11 - DNA, 73 bp, Unknown
  Alanine tRNA sequence
gggggattagctcaaatggtagagcgctcgcttagcatgcgagaggtagcgggatcgatgcccgcatcctcca

SEQ ID NO: 12 - DNA, 74 bp, Unknown
  Threonine tRNA sequence
ggcgccgtggcttagttggttaaagcgcctgtctagtaaacaggagatcctgggttcgaatcccagcggtgcct

SEQ ID NO: 13 - DNA, 74 bp, Unknown
  Isoleucine tRNA sequence
ggctggttagttcagttggttagagcgtggtgctaataacgccaaggtcgtgggttcgatccccatatcggcca